(19) United States
(12) Patent Application Publication
(10) Pub. No.: US 2025/0223322 A1
(43) Pub. Date: Jul. 10, 2025

(54) PARASPORIN EXPRESSION VECTOR AND PHARMACEUTICAL COMPOSITION

(71) Applicants: NPT Co.,Ltd., Tokyo (JP); The University of Tokyo, Tokyo (JP)

(72) Inventors: Kenichiro HARA, Tokyo (JP); Miyako SAGARA, Tokyo (JP); Kenzaburo TANI, Tokyo (JP); Shohei MIYAMOTO, Tokyo (JP)

(21) Appl. No.: 19/019,760

(22) Filed: Jan. 14, 2025

Related U.S. Application Data
(63) Continuation of application No. PCT/JP2023/026163, filed on Jul. 15, 2023.

(30) Foreign Application Priority Data
Jul. 15, 2022 (JP) 2022-114410

(51) Int. Cl. C07K 14/325; A61K 38/00; A61P 35/00; C12N 15/86

(52) U.S. Cl. CPC C07K 14/325; A61P 35/00; C12N 15/86; A61K 38/00; C12N 2770/32343

(57) ABSTRACT

There are provided an expression vector into which a gene encoding parasporin (hereinafter abbreviated as “PS”) is inserted that can realize a pharmaceutical composition applicable to the treatment of cancer; a cell into which the expression vector is introduced; a method for expressing PS, a pharmaceutical composition; and a transformant. The expression vector contains a gene encoding parasporin. The expression vector includes viral vectors into which the gene is inserted, and the viral vectors express a PS protein. The gene encoding a PS protein has the full length or a part of PS gene, which includes PS-like genes encoding proteins that have no hemolytic properties against red blood cells and that exhibit selectively cytotoxicity to cancer cells among the Cry proteins produced by Bt bacteria and/or Bt related bacteria.

### TECHNICAL FIELD

The present invention relates to an expression vector, a cell having the expression vector introduced therein, a method for expressing parasporin, a pharmaceutical composition, and a transformant.

### BACKGROUND ART

Parasporin (hereinafter abbreviated as “PS” in some parts) is a cell disruption protein produced by the microorganism Bacillus thuringiensis (hereinafter abbreviated as “Bt bacteria”), and is defined as “a protein that has no hemolytic properties against red blood cells and exhibits cytotoxicity selectively to cancer cells among the Cry proteins produced by Bt bacteria and related bacteria” (Patent Document 1). In general, four types of Cry proteins, PS 1 to 4, that exhibit PS activity have been so far reported. PS precursors produced by Bt bacteria are said to be partially degraded by proteases to become active PS, and pore formation and apoptosis induction have been observed in cancer cells supplemented with purified PS. In addition, among PS, the cytotoxicity of PS2 to cancer cells is said to be higher than that of other PS.

The development of gene therapy drugs for cancer using genetically modified viruses is being actively carried out. For example, oncolytic virus therapy is a treatment that disrupts cancer cells using the cytocidal effect of specific viruses, and so far, herpes simplex virus, adenovirus, measles virus, coxsackie virus, etc. have been reported as typical oncolytic viruses. However, since a single drug has a limited response rate, a foreign gene (for example, a cytokine gene) is loaded into the viral genome to improve the cytocidal effect by synergy with an immune response.

### CITATION LIST

### Patent Literature

Patent Literature 1: Japanese Patent Laid-Open No. 2005-263728

### SUMMARY OF INVENTION

### Technical Problem

The purpose of the present invention is to provide an expression vector into which a gene encoding PS is inserted that can realize a pharmaceutical composition applicable to the treatment of cancer, a cell into which the expression vector is introduced, a method for expressing PS, a pharmaceutical composition, and a transformant.

### Solution to Problem

The present inventors have developed a PS gene-loaded viral vector for the purpose of improving the cytocidal effect of oncolytic viruses. The present invention not only enables the improvement of the cytocidal effect of oncolytic viruses for the treatment of cancer, but also enables the expression of PS in target cancer cells to obtain the cytocidal effect by inserting PS gene into the non-oncolytic viral genome and by introducing the virus into target cancer cells as an expression vector. Furthermore, the PS-expressing cells obtained by introducing the above-mentioned expression vector into non-cancer cells can be applied to the treatment of cancer.

An expression vector of the present invention contains a gene encoding the full length or active region of PS.

A cell of the present invention is one into which an expression vector is introduced.

A method for producing cells of the present invention includes a step of introducing the expression vector of the present invention into cells.

A method for expressing PS of the present invention is to express PS in eukaryotic cells using an expression vector.

A pharmaceutical composition of the present invention contains the full length or active region of PS gene.

A transformant of the present invention contains the expression vector of the present invention.

### Advantageous Effect of Invention

It is possible to realize a pharmaceutical composition applicable to the treatment of cancer by using an expression vector (such as a viral vector) into which a gene encoding PS is inserted.

### BRIEF DESCRIPTION OF DRAWING

FIG. 1 shows the cytocidal effect.

### DESCRIPTION OF EMBODIMENTS

Hereinafter, preferred embodiments for carrying out the present invention are described in detail.

Expression vectors according to the embodiments contain the full length or active region of PS gene. The expression vectors include viral vectors into which PS gene is inserted, and these expression vectors can express PS in the target cells.

Genes encoding PS have the full length or active region of PS gene, and can include PS-like genes encoding proteins that have no hemolytic properties against red blood cells and that exhibit cytotoxicity selectively to cancer cells among the Cry proteins produced by Bt bacteria and/or Bt related bacteria.

Cells according to the embodiments have an expression vector introduced therein. A known method can be employed for the introduction of the expression vector into cells. Expression vectors contain, for example, an artificially synthesized nucleic acid that can express a gene encoding a viral protein necessary to give rise to a virus. Expression vectors may contain a transcriptional regulatory control sequence functionally linked to a polynucleotide containing the full length or active region of the inserted PS gene. The transcriptional regulatory control sequence referred to here is, for example, a promoter for initiating transcription, an expression control element for enabling binding of ribosomes to the transcribed mRNA.

Viruses belonging to Adenoviridae, Picornaviridae, Herpesviridae, Paramyxoviridae, Parvoviridae, Reoviridae, Poxviridae, Retroviridae, Rhabdoviridae, etc. can be used as a viral vector among expression vectors. A gene encoding a viral protein necessary to give rise to the above-mentioned viruses can be prepared by artificial synthesis.

Examples of the cells into which the expression vector is introduced include eukaryotic blood cells (T cells, B cells, monocytes, dendritic cells, neutrophils, platelets, and erythroblasts), stem cells (hematopoietic stem cells, mesenchymal stem cells, muse cells, embryonic stem cells, and induced pluripotent stem cells), and cancer cells.

The method for producing cells according to the embodiments includes a step of introducing the expression vector according to the embodiments into the cells according to the embodiments.

The method for expressing PS according to the embodiments can be a method expressing PS in eukaryotic cells using an expression vector according to the embodiments, and can be a method expressing PS and PS-like protein in eukaryotic cells using an expression vector.

The pharmaceutical composition according to the embodiments may contain the full length or active region of PS gene. The pharmaceutical composition according to the embodiments may contain the full length or active region of PS-like gene. The pharmaceutical composition according to the embodiments can be used for the treatment of cancer. The pharmaceutical composition can be a formulation for topical and systemic administration. The pharmaceutical composition may contain cells that have the full length or active region of PS gene or PS-like gene.

Further, the pharmaceutical composition according to the present embodiments can be of various dosage forms and various administration routes can be taken. That is, the pharmaceutical composition according to the present embodiments can be a topical preparation and a preparation for systemic administration. For example, it can be an injection or an intravenous agent, and can be administered intratumorally, intravenously, intrathoracically, or intraperitoneally depending on the type of cancer. Particularly, for many gastrointestinal cancers such as esophageal cancer and colon cancer, the pharmaceutical composition can be directly injected into the tumor tissue while observing the tumor tissue using an endoscope, etc. In this case, the injection site can be confirmed using an endoscope, etc., which has the advantage of making it easier to deal with bleeding. Alternatively, the pharmaceutical composition can be administered orally, intramuscularly, subcutaneously, rectally, vaginally, nasally, etc.

The pharmaceutical composition according to the present embodiments may contain a carrier, a diluent, an adjuvant, etc. Examples of the preferred carrier include, extracellular vesicles such as exosomes, liposomes, or lipid nanoparticles such as nanoemulsions, micelles, and solid lipid particles. Liposomes include combinations of lipids and steroids or steroid precursors that contribute to membrane stability. In this case, examples of the lipid include phosphatidyl compounds, such as phosphatidylglycerol, phosphatidylcholine, phosphatidylserine, sphingolipids, phosphatidylethanolamine, cerebroside, and ganglioside. By using the above-mentioned carriers, the immune response of the host can be reduced.

Examples of the diluent include demineralized water, distilled water, and physiological saline solution. Examples of the adjuvant include vegetable oils, cellulose derivatives, polyethylene glycol, and fatty acid esters.

In the case of oral administration, the pharmaceutical composition may contain a sweetener, a disintegrant, a diluent, a coating agent, a preservative, etc.

The pharmaceutical composition according to the present embodiments is administered in such a way as to be in an amount sufficient to treat cancer. The dosage is determined based on the patient's weight, age, sex, size of tumor tissue, etc. The pharmaceutical composition can be administered in a single dose or in a plurality of doses. Further, the pharmaceutical composition can be continuously administered as a sustained-release formulation.

The pharmaceutical composition according to the present embodiments can be used in combination with an anticancer agent. By using the pharmaceutical composition in combination with an antineoplastic agent having a mechanism of action different from that of the pharmaceutical composition, an improvement in the antitumor effect can be expected. The antineoplastic agent is preferably used for, but not particularly limited to, the treatment of head and neck cancer, esophageal cancer, lung cancer, malignant mesothelioma, breast cancer, stomach cancer, pancreatic cancer, prostate cancer, ovarian cancer, uterine cancer, colorectal cancer, colorectal cancer, leukemia, etc. Specific examples of antineoplastic agents include molecular targeted drugs, alkylating agents, metabolic antagonists, anticancer antibiotics, plant alkaloids, platinum preparations, hormone agents, and immune checkpoint inhibitors.

The transformant according to the embodiments includes the expression vector according to the embodiments.

### Example 1

Hereinafter, an example is described.

Wild-type coxsackievirus group B type 3 (hereinafter abbreviated as “CVB3”) was prepared by the method disclosed in International Publication No. WO2018/194089 and Japanese Patent No. 6832422. A gene encoding active PS2Aa1 was inserted into the CVB3 genome to prepare PS2 gene-loaded CVB3 (PS2-CVB3). A polynucleotide containing a gene encoding active PS2Aa1 is inserted into an expressible region of the CVB3 genome. The prepared PS2-CVB3 was infected into H1299 cells (non-small cell lung cancer cells, WT-CVB3-sensitive cells), TE-9 cells (esophageal cancer cells, WT-CVB3-insensitive cells), and BT-20 cells (triple-negative breast cancer cells, WT-CVB3-insensitive cells), and the cytocidal effect was evaluated by the crystal violet staining method. Two types of PS2-CVB3 (10th and 11th) were prepared, each differing in the insertion site of the gene encoding active PS2Aa1.

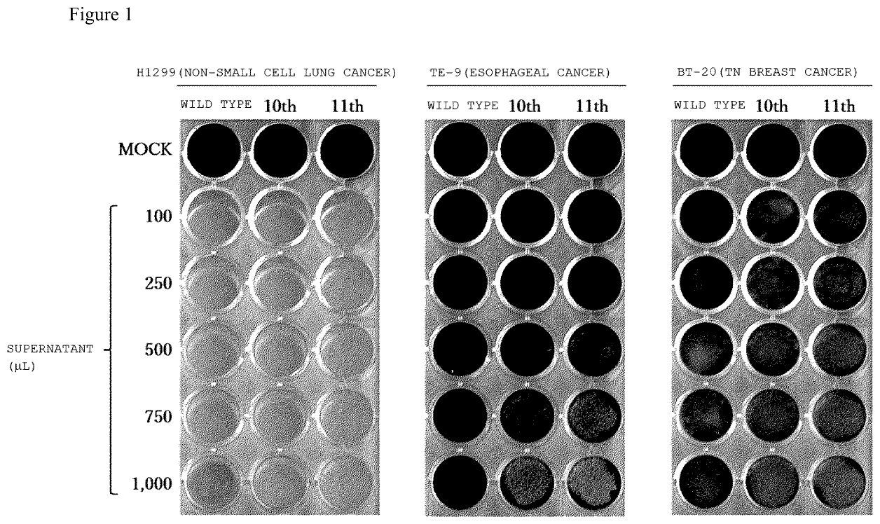

As a result, as shown in FIG. 1, PS2-CVB3 showed a significant cytocidal effect against two types of WT-CVB3-insensitive cells, and it was demonstrated that the cytocidal effect of wild-type CVB3 was improved by expressing active PS2Aa1 in cancer cells.

The present embodiments can be modified in various ways within the scope of the present invention.

1. An expression vector comprising a gene encoding parasporin.

2. The expression vector according to claim 1, wherein the expression vector comprises a viral vector into which the gene is inserted, and wherein the viral vector expresses a parasporin protein.

3. The expression vector according to claim 1, wherein the gene encoding parasporin is the full length or a part of parasporin gene, and wherein the expression vector comprises parasporin-like genes encoding proteins that have no hemolytic properties against red blood cells and that exhibit selectively cytotoxicity to cancer cells among the Cry proteins produced by Bt bacteria and/or Bt related bacteria.

4. A cell into which the expression vector according to claim 1 is introduced.

5. A method for producing cells, comprising a step of introducing the expression vector according to claim 1 into cells.

6. A method for expressing PS in eukaryotic cells using the expression vector according to claim 1.

7. A method for expressing parasporin and a parasporin-like protein in eukaryotic cells using the expression vector according to claim 3.

8. A pharmaceutical composition comprising the full length or active region of parasporin gene.

9. The pharmaceutical composition according to claim 8, wherein the pharmaceutical composition comprises the full length or active region of parasporin-like gene.

10. The pharmaceutical composition according to claim 8, wherein the pharmaceutical composition is used for the treatment of cancer.

11. The pharmaceutical composition according to claim 8, wherein the pharmaceutical composition is for topical and systemic administration.

12. A transformant comprising the expression vector according to claim 1.
